(19) United States
(12) Patent Application Publication
(10) Pub. No.: US 2025/0319149 A1
(43) Pub. Date: Oct. 16, 2025

(54) COMPOSITIONS FOR TREATING IMBALANCES OF THE GUT MICROBIOTA

(71) Applicant: Provexis Natural Products Limited, Reading (GB)

(72) Inventors: Ruedi Duss, Kaiseraugst (CH); Robert Steinert, Kaiseraugst (CH); Ateequr Rehman, Kaiseraugst (CH)

(73) Assignee: Provexis Natural Products Limited, Reading (GB)

(21) Appl. No.: 18/868,943

(22) PCT Filed: Jun. 7, 2023
(86) PCT No.: PCT/GB2023/051483

§ 371 Date: Nov. 25, 2024

(30) Foreign Application Priority Data
Jun. 7, 2022 (GB) 2208323.2

(51) Int. Cl. A61K 36/81; A61K 45/06; A61P 1/00

(52) U.S. Cl. CPC A61K 36/81; A61K 45/06; A61P 1/00

(57) ABSTRACT

The present invention relates to uses of a tomato extract, and compositions containing tomato extracts that are formulated for delivery to the large intestine, for modulating the gut microbiota to confer a health benefit in a subject. The tomato extract can be used improve resistance to infection in the gut, reduce inflammation in the gut, treat or minimise inflammatory Bowel Disease or treat or minimise irritable bowel syndrome. The extracts may also be used to treat conditions associated with an imbalance of the gut microbiota such as anxiety, depression, Type II diabetes, non-alcoholic fatty liver disease, obesity or cognitive dysfunction.

The present invention relates to uses of a tomato extract and compositions containing tomato extracts for delivery to the large intestine to treat conditions associated with an imbalance of the gut microbiota.

### BACKGROUND

Emerging evidence suggests that the gut microbiota has a critical role in maintaining host health and the pathogenesis of many diseases or conditions. Targeted modulation of the gut microbiota has been suggested as a preventative and/or novel therapeutic approach for several diseases, including obesity, type 2 diabetes (T2DM), and cardiovascular or intestinal inflammatory disease (e.g. Inflammatory Bowel Disease or Irritable Bowel Syndrome). Although several nutritional regimes are available today, including pre and probiotics, their full potential remains unexploited partly due to disagreement existing on a clear definition of prebiotics and probiotics, which has resulted in misinformation and confusion among consumers, researchers, and industry, particularly in the case of prebiotics. To foster an appropriate use of the term ‘prebiotic’, the International Scientific Association of Probiotics and Prebiotics (ISAPP) published a consensus paper in 2017, defining a prebiotic as “a substrate that is selectively utilized by host microorganisms conferring a health benefit”.

A few prebiotics have been introduced on to the market that are positively correlated with health benefits. These include fructans (fructooligosaccharides (FOS) and inulin) and galactans (galactooligosaccharides or GOS). However, many other compositions have been sold as prebiotics and have been asserted to have an impact on host microorganisms although the challenge remains to clearly demonstrate that these candidates confer a host health benefit.

It is therefore an object of the present invention to provide further compositions with demonstrable prebiotic activity that will benefit health.

### SUMMARY OF THE INVENTION

The inventors have established that water-soluble tomato extracts (WSTE) are effective for modulating the gut microbiota in a subject's body and such extracts are therefore useful as a prebiotic for treating conditions caused by or impacted by the gut microbiota, or an imbalance, of the gut microbiota.

According to a first aspect of the invention there is provided a water-soluble tomato extract with activity for inhibiting platelet aggregation for use in modulating the gut microbiota to confer a health benefit in a subject.

The health benefit conferred by WSTE may be one that directly impacts on the digestive system. For instance, WSTE may benefit a subject in at least one of the following ways:
(A) improving digestion;
(B) improving resistance to infection in the gut;
(C) reducing inflammation in the gut;
(D) treating or minimising inflammatory Bowel Disease (IBD); or
(E) treating or minimising irritable bowel syndrome (IBS).

Alternatively, the health benefit conferred by WSTE may be to address conditions whose pathology is caused or aggravated by compounds released by the gut microbiota into the bloodstream and whereby WSTE modulates plasma levels of such compounds by modulating the gut microbiota. For instance, WSTE also benefits a subject by:
(A) treating or preventing anxiety and/or depression;
(B) treating or preventing Type II diabetes;
(C) treating or preventing non-alcoholic fatty liver disease;
(D) Treating or preventing obesity;
(E) preventing or reducing inflammaging (or inflamm-aging);
(F) Immune disorders; and
(G) Improving or maintaining cognitive function and brain health.

According to a second aspect of the invention there is provided a method of modulating the gut microbiota of a subject to confer a health benefit in the subject comprising administering a water-soluble tomato extract with activity for inhibiting platelet aggregation to a subject in need of such treatment.

The inventors were surprised to find that WSTE has efficacy for modulating the gut microbiota and this led them to realise that WSTE may be formulated in novel ways to form compositions of benefit to human health.

According to a third aspect of the invention there is provided a composition formulated for delivery of a water-soluble tomato extract with activity for inhibiting platelet aggregation to the large intestine of a subject comprising the water-soluble tomato extract and at least one excipient that promotes delivery of the water-soluble tomato extract to the large intestine.

According to a fourth aspect of the invention there is provided a composition comprising a water-soluble tomato extract with activity for inhibiting platelet aggregation and at least one further prebiotic.

According to a fifth aspect of the invention there is provided a composition comprising a water-soluble tomato extract with activity for inhibiting platelet aggregation and a probiotic.

WSTE may be given to any mammalian subject and has utility in treating animals of veterinary interest. However, it is preferred that the subject is a human subject.

### BRIEF DESCRIPTION OF THE DRAWINGS

The summary, as well as the following detailed description, is further understood when read in conjunction with the appended drawings. For the purposes of illustrating the disclosed compositions and methods, there are shown in the drawings exemplary embodiments of the compositions and methods; however, the compositions and methods are not limited to the specific embodiments disclosed. In the drawings:

FIG. 1, is a flow diagram illustrating the experimental setup of Example 3.

FIG. 2, is a Bar chart of mean absolute changes in (A) plasma and (B) urine TMAO from baseline to end of the intervention in Fruitflow (a preferred WSTE) and placebo group as discussed in Example 3.

FIG. 3: represents a principal component analysis (PCoA) based on A) Bray Curtis and B) Jaccard distance matrixes of Fruitflow and placebo groups at baseline and the end of the intervention as discussed in Example 3. Ellipses represent an 80% confidence interval. Lines connect samples from the same participants. Density plots show the projection of PCoA points onto the PC1 and PC2 axis. Filled circles and circles represent data from Fruitflow (WSTE) treated subjects at the start and end of the trial respectively. Filled triangles and triangles represent data from placebo treated subjects at the start and end of the trial respectively.

FIG. 4: is a Volcano plot representation of differential abundant Operatuibal taxonomic units (OUT) s between time points at baseline and the end of intervention within groups in (A) Fruitflow and (B) placebo and between groups at (C) species level and (D) genus level as discussed in Example 3. The X-axis position of each point represents effect size differences at the end of the intervention. The horizontal line represents the unadjusted p-value cut-off at 0.05. Points above the effect size cut-off (0.20) and p-value cut-off have different symbol and/or are annotated within the text as follows. dots within Box I represent non-significant taxa; dots within boxes II in FIGS. 4A, 4B and 4C represent taxa above p-value cut-off; dots within boxes III in FIGS. 4D represent taxa above the effect size cut-off; triangles represent taxa above effect size and p-value cut-off.

FIG. 5: illustrates untargeted metabolomics showing (A) PLS-DA 2D plot of the multivariate analysis of plasma samples collected after Fruitflow interventions (triangles) and control samples (circles) (R2=0.432, Q2=−0.766); and (B) the 15 most essential bins driving the discrimination between Fruitflow and control samples as discussed in Example 3.

FIG. 6: is a bar chart of mean absolute changes in plasma LPS from baseline to end of the intervention in Fruitflow and placebo group as discussed in Example 3.

### DETAILED DESCRIPTION

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Reference to a particular numerical value includes at least that particular value, unless the context clearly dictates otherwise. When a range of values is expressed, another embodiment includes from the one particular value and/or to the other particular value. Further reference to values stated in ranges, include each and every value within that range. All ranges are inclusive and combinable.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

The following abbreviations are used herein: cardiovascular disease (CVD); Active Pharmaceutical Ingredient (API); water-soluble tomato extract (WSTE); trimethylamine-N-oxide (TMAO); trimethylamine (TMA); lipopolysaccharides (LPS); short chain fatty acids (SCFA); Operational Taxonomic Unit (OTU); type 2 diabetes (T2DM); Inflammatory Bowel Disease (IBD); Irritable Bowel Syndrome (IBS); non-alcoholic fatty liver disease (NAFLD); and Colony Forming Units (CFUs).

As used herein, the singular forms “a,” “an,” and “the” include the plural.

When values are expressed as approximations, by use of the antecedent “about,” it will be understood that the particular value forms another embodiment. Furthermore, the term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

As used herein, “treating” and like terms refer to reducing the severity and/or frequency of symptoms, eliminating symptoms and/or the underlying cause of said symptoms, reducing the frequency or likelihood of symptoms and/or their underlying cause, delaying, preventing and/or slowing the progression of a condition and improving or remediating damage caused, directly or indirectly, by the condition.

As used herein, the phrase “therapeutically effective dose” refers to an amount of a composition comprising WSTE as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, reducing TMAO or LPS plasma levels.

A “pharmaceutically acceptable vehicle” may be any physiological vehicle known to those of ordinary skill in the art useful in formulating pharmaceutical compositions.

WSTE is known to have a number of health benefits including the treatment of cardiovascular conditions (e.g. see WO 2010/049707). The mechanism of action of WSTE was thought to be by directly modulating pathophysiology of a subject being treated and to date no prebiotic activity has been associated with WSTE. The inventors were inspired to investigate the effect of WSTE on the gut microbiome when they noticed that plant polyphenols have been described as being potential prebiotics and they realised that WSTE contains such polyphenols.

It is understood that an estimated 90-95% of plant polyphenols are not absorbed in the small intestine and reach the colon where they undergo extensive biotransformation by the gut microbiota (Clifford (2004) Planta Med 70:1103-11014)).

It has been hypothesized that health benefits associated with polyphenol consumption may depend on microbial utilization and metabolites produced rather than on the parent compound (Duenas et al. (2015) BioMed Res Int 2015:850902). For instance, Chen et al. (2016) (mBio American Society of Microbiology 2016; e02210-e2215) have shown that polyphenols may attenuate trimethylamine-N-oxide (TMAO)-induced atherosclerosis by remodelling gut microbiota and thus lowering TMAO synthesis. TMAO is derived from trimethylamine (TMA), a microbial metabolite produced by various taxa of the gut microbiota primarily from dietary phosphatidylcholine and L-carnitine, which are commonly found in red meat, cheese, and eggs. TMA is absorbed via the intestinal epithelium and further transported to the liver, which is subsequently converted into TMAO. TMAO is known for its proinflammatory and proatherogenic activities, and higher baseline levels of TMAO have been linked to major adverse cardiovascular events.

The inventors therefore conducted research as outlined in the examples and were surprised to find that WSTE was able to transit through the stomach and small intestine to the microflora found in the large intestine; was able to modulate TMAO and LPS production that is mediated by the gut microbiota (discussed below); and also influenced the beta diversity of the gut microbiota (discussed below).

These surprising discoveries lead them to realise that WSTE had prebiotic activity and could be used to confer health benefits in a subject as discussed in the Summary of the Invention and below.

### Water-Soluble Tomato Extracts Used According to the Invention

The water-soluble tomato extract (WSTE) used according to the invention comprises substantially heat-stable colourless water-soluble compounds with activity for preventing platelet aggregation having a molecular weight of less than 1,000 daltons.

The extract may be derived from the flesh of a peeled tomato fruit and/or the juice surrounding the pips of a tomato fruit.

The extracts may essentially be derived from the juice of a tomato fruit which is then further processed as discussed herein.

The extract may be an active fraction which is isolatable from tomato and is preferably characterised in that it:
(a) is colourless or straw-coloured;
(b) contains water soluble compounds;
(c) consists of components having a molecular weight of less than 1000; and
(d) contains one or more nucleosides having platelet aggregation inhibiting activity.

Preferred WSTE comprise at least one of:
(a) a glycosylated phenolic acid or a phenolic ester, or derivatives thereof;
(b) a glycosylated flavonoid; and
(c) a nucleoside.

The glycosylated phenolic acid or phenolic ester may be a glycosylated cinnamic acid or derivative thereof. Such a glycosylated cinnamic acid or derivative thereof may be selected from the group comprising Caffeoyl-4-O-quinic acid, Caffeoyl-4-O-glucoside, Coumaroyl-4-O-glycoside (glue/gal) and Coumaroyl-4-O-glycoside (disaccharide).

The glycosylated phenolic acid or phenolic ester may be selected from: Caffeic acid glucoside; p-Coumaric acid hexose/dihydrokaempferol hexose; Ferulic acid glycoside; and a p-Coumaric acid derivative.

The glycosylated flavonoid may be Quercetin-3-O-glucoside or Rutin.

The nucleoside may be selected from the group comprising AMP, Uridine, Adenosine, Guanosine or GMP.

In preferred embodiments the WSTE is any extract with activity for preventing platelet aggregation that is disclosed in WO 99/55350 or WO 2010/049707.

Preferred WSTEs, including those disclosed in WO 99/55350 or WO 2010/049707, are lycopene free or substantially lycopene free. By “substantially lycopene free” we mean the WSTE is for the most part, free of lycopene that is found in most tomato juices. By this we mean that there is less than 10% w/w, preferably less than 5% w/w and most preferably less than 1% w/w of lycopene (for example 0.75%. 0.5%, 0.25% or 0.1%) in the WSTE than would be found in the equivalent weight of tomato fruit or tomato juice. In a preferred embodiment the WSTE comprises no, or just trace amounts, of Lycopene.

WO 99/55350 and WO 2010/049707 also disclose preferred methods for manufacturing tomato extracts (WSTE) that may be used according to the present invention.

WO 2010/049707 discloses most preferred methods for producing WSTE that may be used according to the present invention. Such water-soluble extracts were found in human trials to have significant efficacy for preventing or reducing platelet aggregation in response to adenosine diphosphate and collagen, and have been marketed, with a European Food Safety Authority authorised health claim in Europe, as a nutritional supplement with health benefits in the cardiovascular area under the brand FRUITFLOW®.

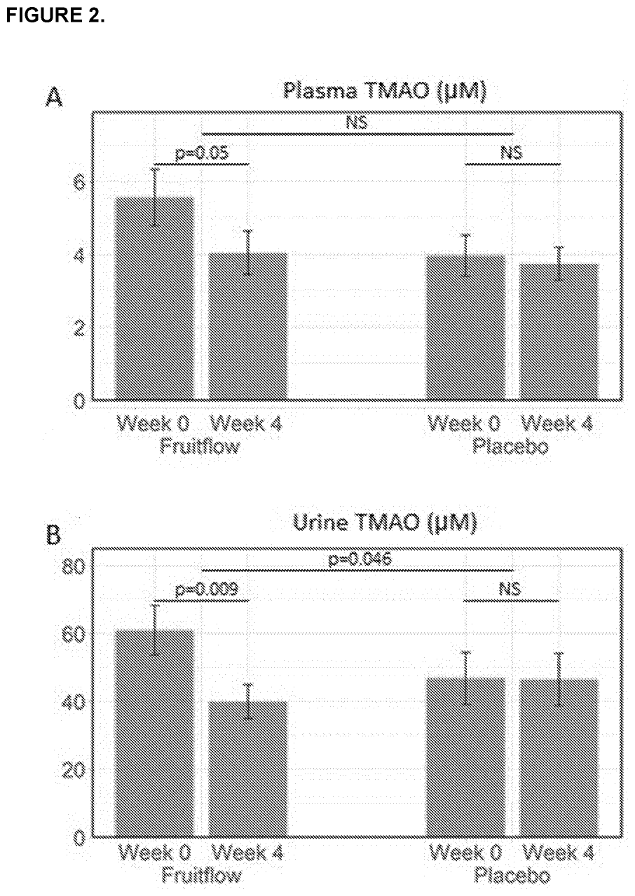

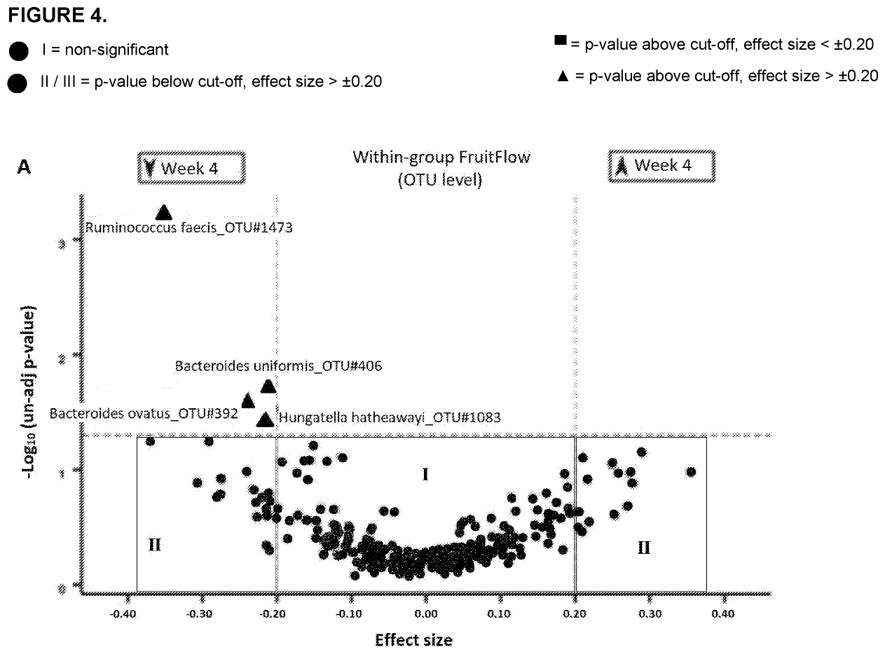

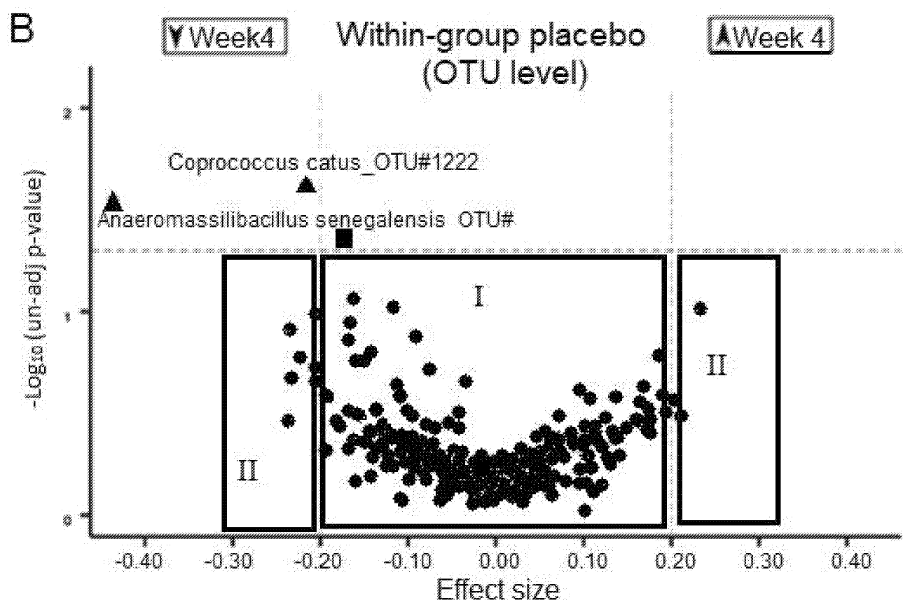

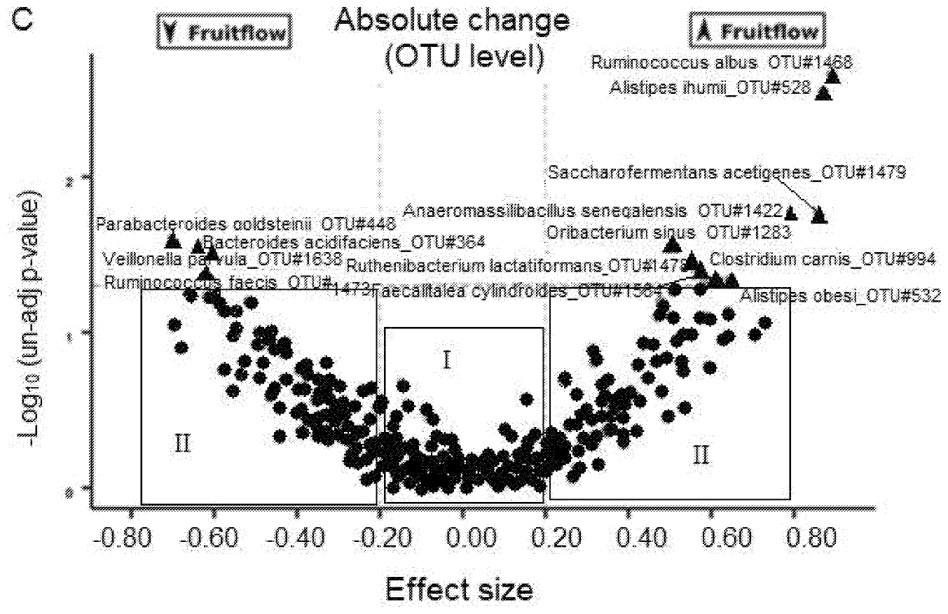

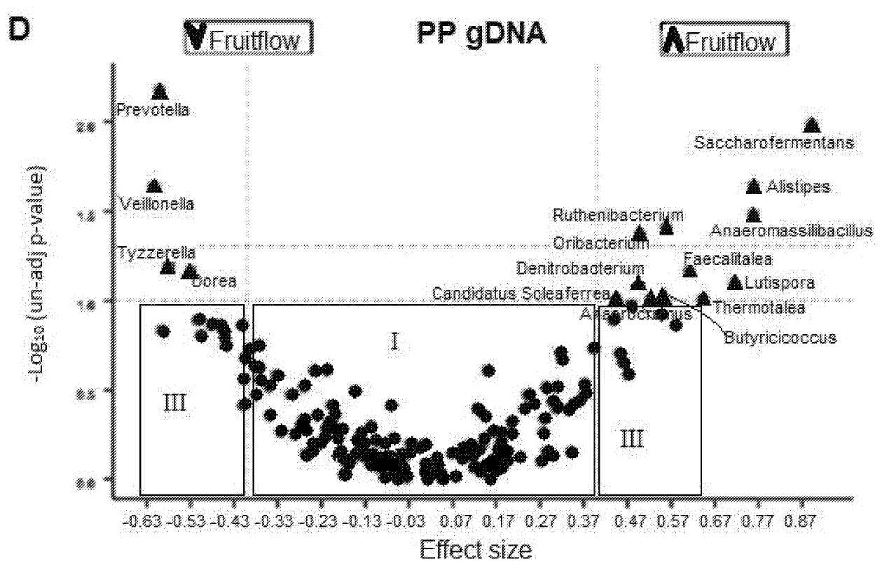

WO 2010/049707 discloses most preferred methods for producing WSTE that may be used according to the present invention, in FIG. 2 (methods for making a liquid/syrup extract from the fruit) and FIG. 4 (methods of processing the extract to make a powder with sugars removed therefrom). These extracts, and the methods of manufacturing them, are incorporated herein by reference. FIG. 2 and FIG. 4 of WO 2010/049707 describe preferred methods of manufacturing two forms of WSTE marketed as FRUITFLOW®. It is most preferred that the WSTE used according to the invention is FRUITFLOW®

### Optional Further Active Ingredients Included in Compositions According to the Invention

### Further Prebiotics

As stated above ISAPP defines a prebiotic as “a substrate that is selectively utilized by host microorganisms conferring a health benefit”. Various prebiotics have been proposed and the inventors have now surprisingly found that WSTE has prebiotic properties and beneficial effects as described herein.

It will be appreciated that the health benefits conferred by WSTE may be enhanced further by including other prebiotics. According to the fourth aspect of the invention a composition may comprise WSTE combined with a further prebiotic.

Preferred further prebiotics used according to the fourth aspect of the invention are fibres and starches that are resistant to mammalian digestion and which may therefore be fermented by the gut microbiome, and especially probiotics, in the large intestine of a subject. Prebiotic fibres are typically complex polysaccharides and may include starches resistant to mammalian digestion; and the soluble fibres inulin, fructooligosaccharides (FOS), and galactooligosaccharides (GOS).

Prebiotics for inclusion in compositions according to the fourth aspect of the invention may be sourced from plants rich in starches that are resistant to mammalian digestion and include green banana, plantain flour, oats, white rice and raw potato.

Prebiotics for inclusion in compositions according to the fourth aspect of the invention may be sourced from plants rich in inulin (e.g. chicory root, Jerusalem artichoke, garlic, onions, leeks, asparagus and ripe bananas).

Prebiotics for inclusion in compositions according to the fourth aspect of the invention may also be sourced from plants rich in FOS such as garlic, shallots, onions, Jerusalem artichoke, burdock root and yacon root.

Prebiotics for inclusion in compositions according to the fourth aspect of the invention may also be sourced from plants rich in GOS such as lentils, chick peas, green peas, lima beans and kidney beans.

### Probiotics

In some embodiments WSTE may be combined with a probiotic and according to the fifth aspect of the invention a composition may comprise WSTE combined with a probiotic.

The human body serves as a host to a wide range of micro-organisms including different strains of bacteria, many of which reside in the gut. Most of these are located in the large intestine and some are considered beneficial to human health. The World Health Organization (WHO) defines probiotics as live micro-organisms which when consumed in adequate amounts confer a health benefit.

An increasing range of health benefits have been attributed to the consumption of probiotics by healthy humans. These include improved digestion, improved resistance to infection and better mood and sleep. Furthermore, probiotics can also deliver benefits for a number of clinical conditions including irritable bowel syndrome (IBS); anxiety and depression, obesity and elevated blood LDL cholesterol. It will therefore be appreciated that in some embodiments the beneficial effects of WSTE may be complemented by also administering a probiotic to a subject.

The selection of bacteria strains for use in a probiotic component of compositions according to the invention is dependent upon the condition to be treated.

The inventors have found that compositions according to the fifth aspect of the invention have general health benefits if they contain at least one bacterial strain selected from: Ruminococcus albus, Alistipes ihumii, Anaeromassilibacillus senegalensis, Saccharofermentans acetigenes, Oribacterium sinus, Clostridium carnis, Ruthenibacterium lactatiformans, Alistipes obesi, Faecalitalea cylindroides, Clostridium methylpentosum, Ruminococcaceae Saccharofermentans, Alistipes, Anaeromassilibacillus and Ruthenibacterium Oribacterium.

Compositions according to the fifth aspect of the invention also have general health benefits if they contain at least one bacterial strain selected from L. Rhamnosus. B. Longum, B. Lactis, B. Bifidum, L. Acidophilus, L. Plantarum, L. Casei, L. Reuteri, B. Infantis, L. Bulgaris, L. Fermentum, L. Paracasei, S. Boulardii, B. Breve, L. Brevis, L. Gasseri, L. Helveticus, S. Thermophillus, Pedicoccus acidilactici and B. Animalis.

In one embodiment the probiotics are selected such that they will benefit gut health and in particular will improve the condition of subjects suffering from digestive disorders such as IBS.

When Billions of Colony Forming Units (CFUs) are referred to herein we mean, the total numbers of probiotics in a composition according to the fifth aspect of the invention. This total may comprise a single bacterial strain or multiple bacterial strains. It will be appreciated that a minimum total number of probiotic CFUs needs to reach the intestines for the probiotic to be efficacious. The probiotics industry often recommends that a dose of probiotic should be in the region of 20 Billion CFUs.

Oral consumption of probiotics requires them to pass through the stomach, which is highly acidic. The stomach may typically contain 0.1 molar hydrochloric acid and can be pH 1 to 2. This stomach acid provides a harsh environment which serves as a chemical barrier and has the function of reducing the risk of gastro-intestinal infection. The low pH of stomach acid can inactivate viruses and kill potentially pathogenic yeasts, moulds and bacteria by lysing their cellular membranes. However, although these positive effects may increase infection resistance, stomach acids also make it difficult for orally ingested probiotic bacteria to survive digestion. Once probiotics reach the small intestine, the near neutral pH (typically in the range 6.8 to 7.4) of the intestines is much more favourable for probiotic survival and growth. It will therefore be appreciated that compositions according to the fifth aspect of the invention may be formulated to reduce the destruction of probiotics in the stomach (e.g. by encapsulation with gastric acid resistant materials).

### Compositions for Oral Administration

The compositions used according to invention may comprise WSTE (and a pre or probiotic as appropriate) without any additional components (e.g. a powder of the WSTE which is used by diluting in a liquid or which is encapsulated). However, in preferred embodiments the WSTE is formulated with other agents, as discussed below, to improve their commercial properties (e.g. to improve delivery, shelf-life, taste and the like).

The compositions of the invention are preferably formulated as a pharmaceutical, nutraceutical, health supplement or food/drink composition for oral administration. For example, they can be formulated as gels, solutions, suspensions, syrups, powders, tablets, capsules, lozenges, snack bars or beverages. Such formulations can be prepared in accordance with methods well known to the art.

The WSTE may be formulated in a syrup or other solution for administration orally, for example as a health drink. One or more excipients selected from sugars, vitamins, flavouring agents, colouring agents, preservatives and thickeners may be included in such syrups or solutions. Tonicity adjusting agents such as sodium chloride, or sugars, can be added to provide a solution of a particular osmotic strength. One or more pH-adjusting agents, such as buffering agents can also be used to adjust the pH to a particular value, and preferably maintain it at that value. Examples of buffering agents include sodium citrate/citric acid buffers and phosphate buffers.

In another embodiment a powder form of WSTE is formulated as a tablet for oral consumption. For tablet formation, the WSTE may be typically mixed with a diluent such as a sugar (e.g. sucrose and lactose); sugar alcohols such as xylitol, sorbitol and mannitol; or modified cellulose or cellulose derivatives such as powdered cellulose or microcrystalline cellulose or carboxymethyl cellulose. The tablets will also typically contain one or more excipients selected from granulating agents, binders, lubricants and disintegrating agents. Examples of disintegrants include starch and starch derivatives, and other swellable polymers, for example crosslinked polymeric disintegrants such as cross-linked carboxymethylcellulose, crosslinked polyvinylpyrrolidone and starch glycolates. Examples of lubricants include stearates such as magnesium stearate and stearic acid. Examples of binders and granulating agents include polyvinylpyrrolidone. Where the diluent is not naturally very sweet, a sweetener can be added, for example ammonium glycyrrhizinate or an artificial sweetener such as aspartame, or sodium saccharinate.

In preferred embodiments the WSTE is formulated as a powder, granules, gels or semisolids for incorporation into capsules. When used in the form of powders, the WSTE can be formulated together with any one or more of the excipients defined above in relation to tablets, or can be presented in an undiluted form. For presentation in the form of a gel or semisolid, the WSTE can be dissolved or suspended in a viscous liquid or semisolid vehicle such as a polyethylene glycol, or a liquid carrier such as a glycol, e.g. propylene glycol, or glycerol or a vegetable or fish oil, for example an oil selected from olive oil, sunflower oil, safflower oil, evening primrose oil, soya oil, cod liver oil, herring oil, etc. These can then be filled into capsules of either the hard gelatine or soft gelatine type or made from hard or soft gelatine equivalents. Soft gelatine or gelatine-equivalent capsules are preferred for viscous liquid or semisolid fillings. In one preferred embodiment, a composition according to the invention is provided in powder form optionally together with a preferred solid (e.g. powdered) excipient for incorporation into capsules, for example a hard gelatine capsule. Compositions according to the fifth aspect of the invention that comprise probiotics are preferably encapsulated such that the probiotic content is protected from gastric acid when transiting through the stomach.

Preferred compositions are for human consumption and may be in the form of gummies, sachet powders, tablets and the like that are retailed by the food and drink industry as dietary supplements.

According to one embodiment of the invention the WSTE is formulated in a powder which may be provided to a subject as a pre-mix to make a drink (when diluted in water or the like) or for mixing by the subject with a food before consumption. Alternatively, premixes may be used by food or drinks manufacturers to produce beverages or foods (e.g. a snack bar) with health benefits as described herein.

A most preferred form is a capsule (which may be hard or soft) for use as a dietary supplement. Purely by way of example, the composition may be a size 00 Vegecaps (LGA, La Seyne-sur-mer. France) capsule comprising 150 mgs WSTE and a suitable vehicle/filler for WSTE. The final weight of each capsule being 600 mg (weight of WSTE plus weight of a filler e.g. tapioca starch).

It will be appreciated that products for use as foods, drinks or dietary supplements may be adapted to form pharmaceutical and nutraceutical compositions comprising WSTE.

### Dose Forms and Dose Regimens

The quantity of WSTE that needs to be administered to a subject will depend upon a number of factors. For instance, when the subject is a human the age, sex and weight of the subject.

The WSTE can be presented in the form of unit dosage forms containing a defined amount of the WSTE. Such unit dosage forms can be selected so as to achieve a desired level of biological activity within the large intestine.

For liquid extracts manufactured according to method 1.1 of Example 1 (see below), the recommended daily dose of the fruit extract according to the invention is between 0.5 g and 20 g and more preferably between 2 g and 7 g. A daily dose may be about 3 g. A typical dosage regime for a human may be from about 70 mg to 285 mg, preferably about 25 mg to 100 mg per kilogram of body weight per day.

In a preferred embodiment a low sugar form of WSTE is used and it is most preferred that powder WSTE manufactured according to method 1.2 of Example 1 (see below) is used. When this is the case, a recommended daily dose may be between 10 mg and 1,500 mg of WSTE; preferably a daily dose will be between 50 mg and 1,000 mg of WSTE and more preferably between about 100 mg and about 500 mg. In one embodiment a subject may receive about 300 mg of WSTE a day and this may be administered as a 300 mg dose form or 2×150 mg dose forms. In a most preferred embodiment a daily dose comprises 2 capsules comprising 150 mg WSTE each.

Purely by way of example, a subject will benefit from improved digestion, improved resistance to infection in the gut, or reduced inflammation in the gut if they consume two capsules (comprising 150 mg WSTE in each capsule) per day. These capsules may be consumed in the morning (e.g. with breakfast).

Subjects may take the capsules for as long as a health benefit is derived from consumption of WSTE. A subject may take WSTE as a prophylactic (to prevent the development of a condition e.g. IBS) or may start to take WSTE to treat a condition (e.g. IBS). Accordingly, compositions according to the invention may be taken as part of a morning health supplement routine (e.g. for 28 days or more).

### Example 1: Preparation of WSTE

WSTE for use in compositions according to the invention was prepared by one of the following protocols:

1.1 A liquid (syrup) extract was prepared following the protocols of Example 2 and FIG. 2 of WO 2010/049707 and is known as FRUITFLOW® 1.

1.2 A powder extract with low sugar content was prepared essentially following the protocols of Example 3 and FIG. 4 of WO 2010/049707 and is known as FRUITFLOW® 2. 150 mg of FRUITFLOW® 2 comprises up to 9 mg nucleoside derivatives, up to 10 mg simple phenolic conjugates (e.g., chlorogenic acid, other caffeic/phenolic acid derivatives), and up to 7 mg flavonoid derivatives, of which at least 2.4 mg are quercetin derivatives.

### Example 2: Composition Comprising WSTE

The WSTE described at 1.2 was mixed with three times (in weight) of tapioca starch filler by conventional means and the mix used to fill size 00 capsules (Vegecaps from LGA, La Seyne-sur-mer, France).

Each capsule comprised 150 mg of the WSTE and 450 mg of tapioca starch filler.

### Example 3

The inventors decided to assess the effect of the composition of Example 2 on the gut microbiome by performing a randomized, double-blind, placebo-controlled cross-over trial to investigate the effect of four weeks of supplementation of 2×150 mg WSTE capsules on plasma and urine TMAO and fecal microbiota as well as plasma and fecal metabolites (including plasma lipopolysaccharides (LPS), bile acids, short-chain fatty acids (SCFA) and other organic acids) and gastrointestinal comfort.

### 3.1 Materials and Methods

### 3.1.1 Study Population

The study population consisted of 40 healthy, overweight, and obese adults (BMI 28-35 kg/m2) aged 35-65 years. The main exclusion criteria were as follows: significant acute or chronic disease; smoking; a history of drug and/or alcohol abuse (more than 2 servings of alcohol/day), pregnancy; antibiotic use within the previous 3 months; major dietary changes in the past 3 months; eating disorders; vegetarians or vegans; enemas; dietary supplements including prebiotics, probiotics, or fibre within 4 weeks before the baseline visit and for the duration of the intervention; chronic medications for active gastrointestinal disorders (unless the product was taken for at least 2 months before screening and the exact dosage was maintained throughout the study) and high habitual intake of tomatoes or tomato-based products as confirmed by a Food Frequency Questionnaire. Participants who were deemed eligible were randomly assigned to an intervention order on a 1:1 basis, where n=20 participants were allocated to the Placebo/WSTE arm (group 1), and n=20 participants to the WSTE/Placebo arm (group 2). Three participants self-withdrew after randomization; the remaining 37 participants completed the study as planned. An independent committee evaluated all participants on a case-by-case basis before unblinding the data to determine inclusion in the per-protocol population. 15 subjects were excluded from the analysis because of the use of concomitant medications such as antibiotics (n=9) and IP compliance below 80% and/or missing critical variables (n=6).

### 3.1.2 Study Design

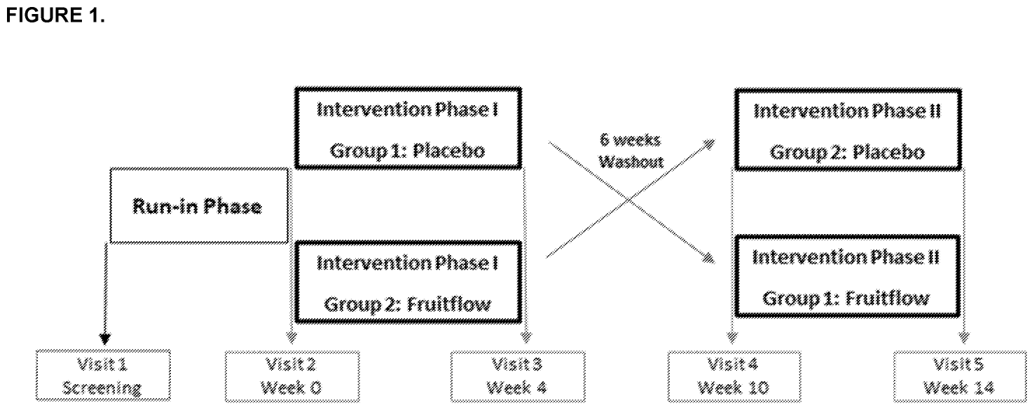

The trial was designed as a randomized, double-blind, placebo-controlled, cross-over trial consisting of 5 visits: the screening visit (visit 1), after a 21-day run-in period, the start of intervention phase 1 (visit 2), end of the 4-week intervention (visit 3), 6-week washout, and start of the second intervention phase (visit 4), end of the 4-week intervention (visit 5) (FIG. 1). During the screening, vital signs were recorded, and a complete medical examination, including medical history and demographic/anthropometric assessment performed. In addition, weekly tomato consumption was queried and a fasting venous blood sample was taken for safety profiling. Participants were provided with a stool collection kit, instructions for collecting and storing the stool sample, and a Bristol Stool Chart (Lewis & Heaton. (1997) Scand J Gastroenterol Taylor and Francis 32:920-924) to be completed at the time of stool collection. At visit 2, participants arrived at the study site and fasted overnight for at least 10 hours. Participants returned the Bristol Stool Chart and stool sample collected and stored at −20° C. in home freezers within 24 hours before the visit. A venous blood sample was collected for safety profiling and analysis of plasma TMAO and lipopolysaccharides (LPS). In addition, a urine sample was collected for analysis of urine TMAO and participants completed a Gastrointestinal Symptom Rating Scale (GSRS) and Food Frequency Questionnaire (FFQ) before they were randomized into one of the two intervention groups. Participants were provided with two stool collection kits and Bristol Stool Charts and instructed to collect a stool sample at home, after 2 weeks, and another sample within 24 hours of their next scheduled visit at week 4. At visit 3, participants again arrived at the study site, fasted overnight, and returned the stool samples and Bristol Stool Chart. Another blood and urine sample was collected, and a Gastrointestinal Symptom Rating Scale was completed. In addition, participants returned any unused study product to assess compliance of intervention phase 1. Participants were then sent home for the 6-week wash-out period before entering intervention phase 2, which followed the same experimental setup as visits 2 and 3.

### 3.1.3 Ethics Statement

The trial was conducted by Atlantia Food Clinical Trials Ltd., in Cork, Ireland in accordance with the Declaration of Helsinki and approved by the Clinical Research Ethics Committee of the Cork Teaching Hospitals. Written informed consent for participation was obtained from all participants.

### 3.1.4 Measurements

Blood & Urine Samples: Blood samples were collected for safety profiling (haematology, chemistry, glucose, high sensitive C-reactive protein (hsCRP), and bilirubin) and analysis of TMAO, lipopolysaccharides (LPS), bile acids (BA), short-chain fatty acids (SCFA), and other organic acids as well as untargeted metabolomics. Urine samples were collected for analysis of urine TMAO.

Safety profiling, including haematology, chemistry, and glucose, was performed by standard clinical laboratory methods in Eurofins Biomnis (Sandyford, Dublin, Ireland).

TMAO and LPS samples were sent to MS-Omics ApS (Bygstubben 9, 2950 Vedbaek, Denmark) for analysis. Plasma LPS analysis was performed by the LC-MS method.

For analysis, bilirubin and hsCRP samples were sent to Eurofins Biomnis (Sandyford, Dublin, Ireland).

Plasma bile acids were extracted from plasma and quantified using a commercially available bile acids assay (Biocrates Life Sciences AG, Austria). SCFA and other organic acids were first extracted from plasma via protein precipitation. Then plasma extracts were derivatized and reaction products extracted by liquid-liquid extraction using dichloromethane. Obtained extracts were finally injected into a UHPLC-MS/MS system for analysis in combined positive and ESI MRM mode.

For untargeted metabolomics, plasma samples were prepared using a previously described method with minor adaptations. (Meilko et al. (2021) Sustain Chem Pharm 22:1000574-). In short, 100 μl of human plasma was mixed with 100 μl of a deuterated phosphate buffer pH=7.3, prepared by adding a PBS tablet (OXOID) in 100 ml of D2O. Maleic acid (0.5 mM) was used as an internal standard, and the samples were subsequently put in 3 mm NMR tubes. All spectra were acquired at 298 K on a Bruker Avance III NMR spectrometer operating at 600 MHz proton Larmor frequency and equipped with a 5 mm TCI cryoprobe. 1D 1H NMR spectra were acquired using a cpmgpr1d pulse sequence, relaxation and D20 delays of 5 and 0.0003 s; 256 scans were accumulated in 36.5 min per spectrum.

Fecal samples: Fecal samples were collected in DNA/RNA Shield™ and fecal collection tubes (Zymo Research, Irvine U.S.A) and delivered on dry ice in −80° C. compatible boxes to BaseClear BV (Leiden, Netherland) for microbiome profiling. In brief, nucleic acid was extracted from fecal samples using ZymoBIOMICS™ DNA Miniprep (Zymo Research Corp., Irvine, CA, USA) kit as per the manufacturer instruction. 16S rRNA gene variable region V3-V4 was amplified by composite primer 341F (5′-CCTACGGGNGGCWGCAG-3′) (SEQ ID No. 1) and 785R (5′-GACTACHVGGGTATCTAATCC-3′ (SEQ ID No. 2) and sequenced using the Illumina MiSeq sequencing platform to generate paired-end sequence reads. Subsequently, reads containing PhiX control signal were removed by aligning the sequence reads against the Phix genome (NC_001422.1) with Bowtie2 (2.2.6) (Langmead & Salzberg (2012) Nat Methods Nature Publishing Group 9:357-9) and removed from further analysis. In addition, reads containing (partial) adapters were clipped (up to a minimum read length of 50 bp). The second quality assessment was based on the remaining reads using the FASTQC quality control tool version 0.11.5. Paired-end sequence reads were collapsed into so-called pseudoreads using sequence overlap with USEARCH version 9.2 (Edgar (2010) Bioinformatics 26:2460-2461). Classification of these pseudoreads is performed based on the results of alignment with SNAP version 1.0.23* (Zaharia et al. (2022) http://arxiv.org/abs/1111.5572) against the RDP database (Cole et al. (2014) Nucleic Acids Res 42: D633-642) for bacterial organisms. Alpha diversity indices (observed species, Chao1, Shannon and Simpson diversity indices) and beta diversity indices (Jaccard and Bray-Curtis) were calculated by implementing mothur version 1.35.1 (https://www.ncbi.nlm.nih.gov/pmc/articles/PMC2786419/).

Stool Consistency and Gastrointestinal Symptoms: Stool consistency was assessed using the Bristol Stool Chart: Type 1 (separate hard lumps, like nuts), Type 2 (sausage-shaped but lumpy), Type 3 (like a sausage but with cracks on its surface), Type 4 (like a sausage or snake, smooth and soft), Type 5 (soft blobs with clear-cut edges), Type 6 (fluffy pieces with ragged edges, a mushy stool), Type 7 (watery, no solid pieces, entirely liquid) (Lewis & Heaton supra). Gastrointestinal symptoms were assessed by a 6-point scale using the GSRS utilizing a 7-point rating scale, depending on the intensity and frequency of GI symptoms experienced during the previous weeks. A higher score indicates more inconvenient symptoms.

### 3.1.5 Investigational Product

150 mg of the WSTE described at 1.2 was combined with 450 mgs of tapioca starch filler and encapsulated using size 00 Vegecaps (LGA, La Seyne-sur-mer, France). 600 mgs of Maltodextrin (encapsulated in the same way) was used as a placebo control (Essential Nutrition Ltd., Brough, UK).

Participants were instructed to consume two capsules orally in the morning at breakfast along with a glass of water for the 28-day intervention period of each intervention phase.

### 3.1.6 Sample Size Calculation and Statistical Analysis

The sample size was determined based on findings from previous studies with nutritional interventions to reduce plasma TMAO. For a power of 80%, the significance level of 5%, and an expected effect size (mean/SD) of 0.5 for WSTE versus placebo, it was calculated that 34 participants were required. To account for potential losses to follow-up, 40 participants were enrolled. To validate the randomization method, the effectiveness of the washout was assessed using paired samples t-tests comparing within-group change from the baseline of phase 1 (visit 2) to the baseline of phase 2 (visit 4). There were no statistically significant changes from phase 1 baseline to phase 2 baseline for any primary or secondary objective within either the WSTE or placebo group. As a result, the WSTE group is a combination of group 2-phase 1 data (visit 2-visit 3) and group 1-phase 2 data (visit 4-visit 5), whereas the placebo group is a combination of group 1-phase 1 data (visit 2-visit 3) and group 2-phase 2 data (visit 4-visit 5).

SPSS IBM V26.0 and R software were used to analyze all data. Unpaired t-tests (or Mann Whitney U Test) were used to compare between-group differences at the baseline of each phase. For the efficacy analysis, paired t-tests (or Wilcoxon Signed Rank Tests) were used to determine (1) if there was a statistically significant within-group change from baseline to end of the intervention in either the Fruitflow group or the placebo group; or (2) if there was a statistically significant between-group difference (i.e., the difference in outcome between the WSTE and placebo group). All data are reported as means±SE. All tests were two-tailed, and differences were considered statistically significant at p≤0.05.

For untargeted metabolomics, all spectra were collected and processed using ACD Labs 2012. All spectral regions containing NMR features were group processed by Intelligent Bucketing (Bucket width=0.02 ppm, width looseness=50%). The resulting bins were analyzed using MetaboAnalyst v4.0 (Chong et al. (2018) Nucleic Acids Res 46:W486-494) after being divided into two groups, i.e., subjects who had received WSTE before blood sampling (group 1, visit 5; group 2, visit 3) and subjects who had not (group 1, visit 2, 3 and 4; group 2 visit 2, 4, and 5). All data were normalized (quantile normalization) and auto-scaled (Pareto scaling) before univariate (t-test) and multivariate analysis (PLS-DA). The Q2 and R2 values for Partial Least Squares Discriminant Analysis (PLS-DA), as well as the Variable Importance in Projection (VIP) plots, were determined by MetaboAnalyst following an established workflow (Macias et al. (2019) Metabolites 9:E201).

For microbial alpha diversity, a paired Wilcoxon test was used to determine statistical significance between group and within groups. For beta diversity, Permutational Multivariate Analysis of Variance (PERMANOVA) was used to quantify differences within and between groups (WSTE vs. placebo) using 9999 permutations. This test evaluates if groups or time points significantly affect the overall gut microbiota composition and structure. Within and between-group changes in the relative abundance of microbial taxa (i.e., the difference in outcome between the WSTE and placebo group) was analyzed using paired Wilcoxon test followed by multiple testing correction with Benjamini-Hochberg (BH). In addition, for between-group changes, raw abundance counts were Centroid Log Ratio (CLR) normalized to ensure that the data is scale-invariant and subcompostionally coherent. Effect size differences for each taxon were calculated using the cohens d function (https://easystats.github.io/effectsize/), an R-based package.

### 3.2. Results

### 3.2.1 Plasma and Urine TMAO

Fasting plasma and urine TMAO: There was a significant within-group change from baseline to end of the intervention in plasma (p=0.05) and urine (p=0.009) TMAO in the WSTE group but not in the placebo group (NS). Between-group changes (i.e., the difference in outcome between the WSTE and placebo group) were significantly different only for urine but not plasma TMAO (p=0.05, FIGS. 2A and B).

### 3.2.2 Fecal Microbiota

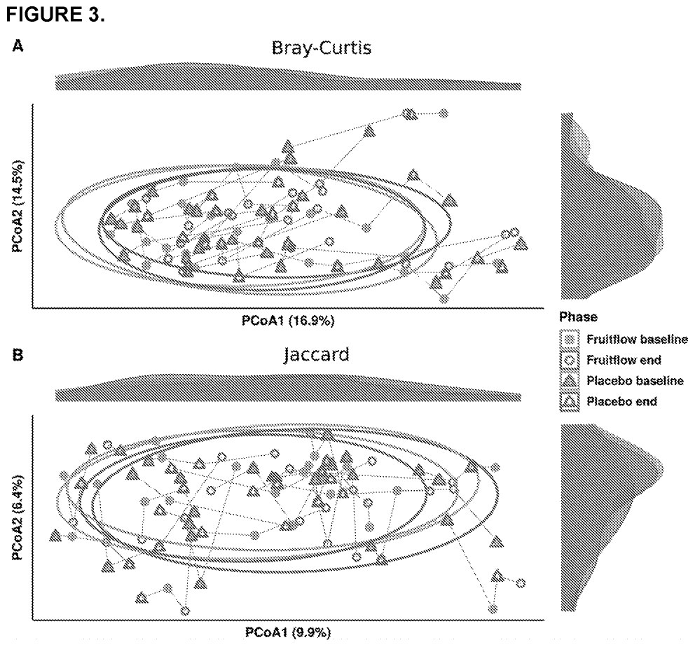

Alpha and beta diversity: No significant within and between-group changes were observed for species diversity and richness using observed species, Chao1, Shannon, and Simpson diversity indices in either the placebo or WSTE group (data not shown). To gain insights into the temporal dynamics of microbial communities, fecal samples were subjected also to a multivariate analysis using Bray-Curtis and Jaccard distance methods. However, no significant shift in beta diversity was observed in global or pairwise PERMANOVA analysis within and between groups. In contrast, a substantial shift in Jaccard Principal Component (PC1) was observed when comparing the end of intervention time points of WSTE and the placebo group (p=0.019, FIG. 3).

Microbial composition: Changes in relative abundances of species-level OTUs within groups from baseline to end of the intervention was performed using ALDE×2. In the WSTE group, four species-level OTUs related to Ruminococcus faecis_OTU #1473 (p=0.0005), Bacteroides uniformis_OTU #406 (p=0.018), Bacteroides ovatus_OTU #392 (p=0.025) and Hungatella hathewayi_OTU #1083 (p=0.036) had lower relative abundance at the end of intervention when compared to baseline (FIG. 4A). In contrast, in the placebo group, three species-level OTUs related to Coprococcus catus_OTU #1222 (p=0.023), Anaeromassilibacillus senegalensis_OTU #1422 (p=0.028), Barnesiella intestinihominis_OTU #428 (p=0.043) were significantly depleted at the end of intervention when compared to baseline (FIG. 4B).

The inventors calculated between-group changes (i.e., the difference in CLR abundance for each species-level OTU between the WSTE and placebo group) using the Wilcoxon test. They found that relative abundances of 17 OTUs significantly differed between groups, of which nine OTUs related to Ruminococcus albus_OTU #1468 (p=0.002), Alistipes ihumii_OTU #528 (p=0.002), Anaeromassilibacillus senegalensis_OTU #1422 (p=0.017), Saccharofermentans acetigenes_OTU #1479 (p=0.017), Oribacterium sinus_OTU #1283 (p=0.027), Clostridium carnis_OTU #994 (p=0.035), Ruthenibacterium lactatiformans_OTU #1478 (p=0.039), Alistipes obesi_OTU #532 (p=0.046), and Faecalitalea cylindroides_OTU #1564 (p=0.046) were elevated significantly in the WSTE group when compared with placebo. In addition, four OTUs related to Parabacteroides goldsteinii_OTU #448 (p=0.025), Bacteroides acidifaciens_OTU #364 (p=0.027), Veillonella parvula_OTU #1638 (p=0.030) and Ruminococcus faecis_OTU #1473 (p=0.039) had significantly lower abundances in WSTE group when compared with placebo (FIG. 4C).

At the genus level, three taxa related to Streptococcus (p=0.005), Rumniococcus (p=0.021), and Hungatella (p=0.039) had significantly lower relative abundance at the end of intervention when compared to baseline in the WSTE group. In contrast, in the placebo group, Anaeromassilibacillus (p=0.028), Alistipes (p=0.038), and Eubacterium (p=0.038) were significantly depleted at the end of intervention when compared to baseline (data not shown).

Furthermore, a comparison of between-group changes revealed a significant decrease in the relative abundance of Prevotella (p=0.007) and Veillonella (p=0.023). At the same time, five taxa related to Saccharofermentans (p=0.01), Alistipes (p=0.023), Anaeromassilibacillus (p=0.033), Ruthenibacterium (p=0.039), and Oribacterium (p=0.042) were significantly increased in the FRUITFLOW group when compared to changes in the placebo group (FIG. 4D).

The inventors did not observe any significant within or between-group changes at the phylum level (data not shown)

### 3.2.3. Plasma Metabolites

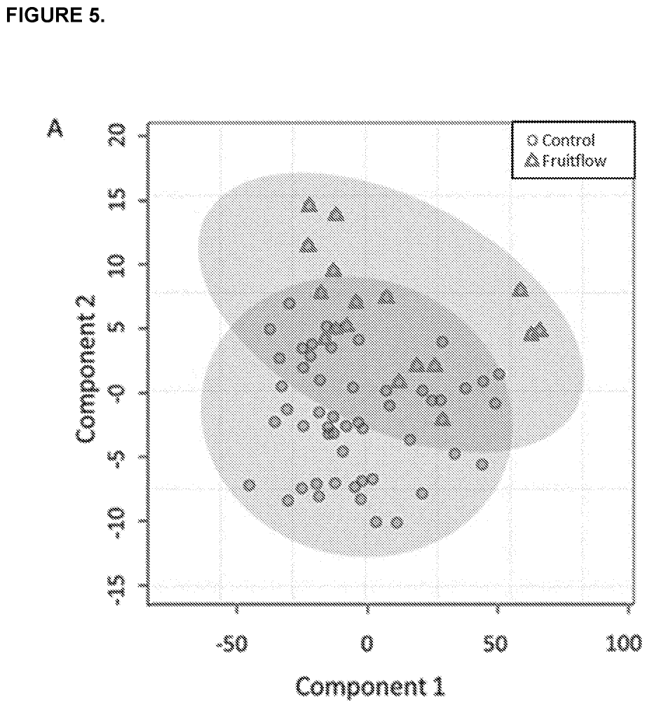

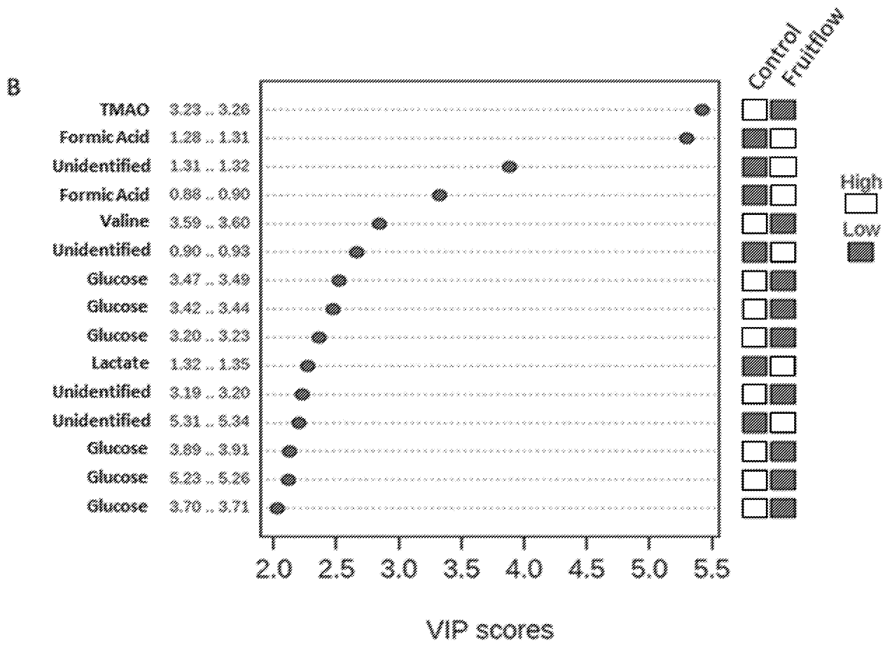

Plasma untargeted metabolomics: Principal component analysis (PCA) of the NMR data shows a clear distinction between plasma samples collected after WSTE intervention and control samples (FIG. 5A). By means of variable importance in projection (VIP), the top 15 ranking features driving this distinction included TMAO, formic acid, valine, glucose, and lactate, with TMAO being the most discriminant metabolite (low in WSTE samples, high in control samples, FIG. 5B).

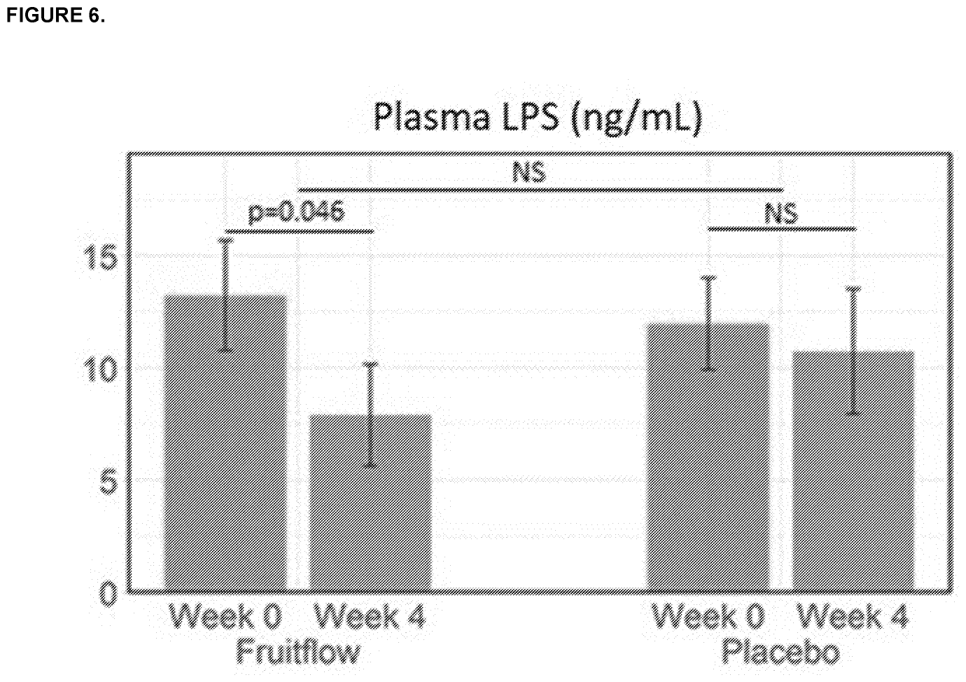

Plasma LPS: Plasma LPS concentrations were significantly lower at the end of the intervention than at baseline in the Fruitflow group (p=0.05) but not in the placebo group. However, there were no significant between-group changes (FIG. 6).

Plasma bile acids: Several bile acids were detected in plasma in both groups, including cholic acid (CA), chenodeoxycholic acid (CDCA), deoxycholic acid (DCA), glycocholic acid (GCA), glycochenodeoycholic acid (GCDCA), glycodeoxycholic acid (GDCA), glycoursodeoxycholic acid (GUDCA), taurochenodeoxycholic acid (TCDCA), taurodeoxycholic acid (TDCA), and ursodeoxycholic acid (UDCA). TCDCA and TDCA increased slightly in both groups, WSTE, and placebo, from baseline to end of intervention (P≤0.05), while CDCA, GCA, GCDCA, and GDCA only increased in the WSTE group (P≤0.05). However, there were no significant between-group changes (data not shown)

Plasma SCFA and other organic acids: Acetate, lactate, pyruvate, and 3-hydroxybutyrate were detected in plasma. A slight but significant within-group increase from baseline to the end of the intervention was observed in the WSTE group for plasma pyruvate. In contrast, plasma acetate was slightly increased in both groups (P≤0.05, respectively). There were no significant between-group changes (data not shown).

### 3.3.4 Fecal Metabolites

Fecal bile acids: Several bile acids were detected in feces in both groups, including CA, CDCA, DCA, GCA. GCDCA, GDCA, glycolithocholic acid (GLCA), GUDCA, lithocholic acid (LCA), taurochenodexycholic acid (TCDC), and TDCA. We observed one significant within-group change; fecal CA increased from baseline to the end of the intervention in the WSTE group but not in the placebo group (P≤0.05). There were, however, no significant between-group changes (data not shown).

Fecal SCFA and other organic acids: Acetate, propionate, butyrate, valerate, isovalerate, 2-methyl butyrate, isobutyrate, and pyruvate were all detected in feces. There was, however, no effect of Fruitflow on fecal SCFAs; only valerate increased from baseline to end of the intervention in the placebo group, and this difference was significant also between groups (P≤0.05, respectively, data not shown).

### 3.3.5 Stool Consistency and Gastrointestinal Symptoms

There were no significant within or between-group changes in stool consistency or gastrointestinal symptoms. Data not shown.

### 3.3.6 Adverse Events and Blood Safety Profiles

There were no serious adverse events (SAEs), or withdrawals due to adverse events (AEs) observed during the study. All AEs were of mild or moderate intensity, and none were deemed to be related to the investigational products by the assigned study clinician. Standard hematology and biochemistry assessment showed no significant within and between-group effects, including glucose, hsCRP, and bilirubin. Data not shown.

### 3.4. Discussion

The inventors found that WSTE significantly decreased fasting blood (−1.51 μM) and urine (−19.09 μM) TMAO when compared to baseline as well as plasma LPS (−5.33 ng/ml), a marker of intestinal permeability and low-grade inflammation. An untargeted metabolomic analysis confirmed the effect of WSTE on plasma TMAO. A clear distinction was observed between plasma samples collected after WSTE interventions and control samples, with TMAO being the top ranking feature driving this distinction.

When analysing fecal microbiota, the inventors found changes in microbial beta, but not alpha, diversity with a significant difference in Jaccard distance-based Principal Component when comparing WSTE vs. placebo. In addition, there were several significant changes in microbial composition with WSTE, such as decreases in Bacteroides, Ruminococccus, and Hungatella related OTUs, which are known for their involvement in TMA/TMAO metabolism, and increases in Alistipes and Clostridia related OTUs, the latter which are well known SCFA producers.

Finally, the inventors found increases in the number of fecal and plasma bile acids and plasma pyruvate but no change in fecal SCFA. No effect of WSTE on gastrointestinal comfort was observed.

### 3.4.1 the Impact of WSTE on Reducing TMAO Levels

TMAO has been established as an independent risk factor for promoting atherosclerosis by stimulating foam cell formation, deregulating enterohepatic cholesterol and bile acid metabolism, and impairing macrophage reverse cholesterol transport (Wang et al. (2011) Nature 472: p57-63; Koeth et al. (2013) Nat Med 19 p576-585; Canyelles et al. (2018) Int J Mol Sci 19:E3228; and Tang et al. (2013) N Engl J Med 368 p1575-1584). Given that the production of TMAO from dietary phosphatidylcholine is dependent on metabolism by the intestinal microbiota (Wang et al., Koeth et al. and Tang et al. supra), gut microbiota-based therapies have been suggested as a novel strategy for preventing and treating cardiovascular disease (CVD). Oral broad-spectrum antibiotics can limit TMAO-induced atherosclerosis (Koeth et al. supra) by suppressing intestinal microbiota; however, side effects and resistance potential restrict their utility. Therefore, the focus is on natural products with characteristics to selectively modulate the gut microbiota to inhibit microbial TMA production and, as a result, lower the risk for CVD. This profile suggests WSTE falls within the definition of a prebiotic and is a selectively utilized substrate for host microorganisms that confer a health benefit.

The inventors do not wish to be bound by any hypothesis but believe the prebiotic benefits of WSTE may, at least in part, be due to the polyphenols contained within the extract. Several natural products rich in polyphenols have been shown recently in animals and humans to lower plasma TMAO levels. However, there are also contradicting reports that some natural products rich in polyphenols raise plasma TMAO levels. For example. Chen et al. (supra) first demonstrated in mice that resveratrol attenuates TMAO-induced atherosclerosis by regulating TMAO synthesis via remodeling gut microbiota. This was confirmed in other studies with experimental rodents as well as in a pilot study in 20 normal-weight subjects showing a significant decrease in serum TMAO (1.87 to 0.66 μM) following four weeks of supplementation with a 300 mg of polyphenol-rich grape pomace (Annunziata et al. (2019) Digital Publishing Institute 11:139-139). In contrast, a recent study in overweight and obese subjects revealed that raspberry consumption of 280 mg/day increased plasma TMAO levels (Franck et al. (2022) Nutrients 14:1656). These prior art reports make it unclear whether or not a skilled person would expect a polyphenol rich extract such as WSTE to raise or lower TMAO levels. Franck et al. (supra) suggested that distinct gut microbial signatures with the higher relative abundance of Actinobacteriota in participants whose TMAO levels increased after the intervention could explain such an effect; however, this required further research. The inventors did not observe increased levels of Actinobacteria in this study. Nevertheless, the inventors believe the data presented herein does correlate a decreased plasma TMAO level caused by WSTE with health benefits.

The mean decrease of 1.51 μM in plasma TMAO for this study was similar to what was observed in the grape extract study by Annunziata and colleagues (supra) (mean reduction of 1.2 μM). Moreover, the observed effects suggest a clinical relevance given the findings of a recent cross-sectional study including 377 patients with acute ischemic stroke and 50 healthy controls. In this study, plasma TMAO levels were higher in patients with ischemic stroke than in healthy controls (median 5.1 vs. 3.0 μmol/L; p<0.001), and every 1-μmol/L increase in TMAO was associated with a 1.13-point increase in the NIH Stroke Scale and 1.69-mL increase in infarct volume after adjustment for vascular risk factors.

The inventors have noted that increased plasma TMAO levels are associated with adversely affecting the conditions contemplated in the Summary of Invention section. The data presented herein (illustrating that WSTE decreases plasma TMAO levels) lead them to realise that WSTE, acting as a prebiotic, can be used to treat, prevent or relieve the symptoms of such conditions. By way of example, WSTE is effective for treating inflammaging and age-related cognitive dysfunction. Studies in mice and rats suggest that aging-induced gut dysbiosis produces higher TMAO, contributing to an increased peripheral and central inflammatory tone resulting in vascular inflammation, oxidative stress, and eventually age-associated endothelial dysfunction and cognitive deficiencies (Li et al. (2018) Aging Cell17:e12768). Furthermore, in humans, cerebrospinal fluid TMAO was higher in individuals with mild cognitive impairment and Alzheimer's disease dementia compared to cognitively-unimpaired individuals (Vogt et al. (2018) Alzheimers Res Ther. 10 (1): 124). Together, this made the inventors realise that, given WSTE decreases plasma TMAO by modulating the gut microbiota, that it also represents a novel therapeutic option for treating inflammaging and neurodegenerative diseases. Furthermore, WSTE may be used to generally promote brain health in subjects of all ages and in particular the elderly.

### 3.4.2 the Impact of WSTE on Gut Microbiota

The inventors were surprised to find WSTE also altered the gut microbiome. These changes were not reflected in microbial alpha diversity indices. However, a surprising change was seen in beta diversity with a significant shift seen in Jaccard PC1, indicating that WSTE consumption substantially affects gut microbial composition. Franck et al. (supra) investigating the effect of raspberry consumption on TMAO (when contrary to the present invention they found an overall increase in plasma TMAO) also found no significant change in alpha diversity indices; however, they did not report beta diversity.

The inventors' observations with regards the change in beta diversity may support the notion that the changes in plasma and urine TMAO are dependent on the changes in gut microbiota caused by WSTE administration. WSTE appears to achieve this by decreasing the proportion of bacteria that produce TMA and/or increasing the proportion of microbes metabolizing TMA efficiently. The inventors observed a significant decrease in the relative abundance of Bacteroides uniformis and Bacteroides ovatus within the WSTE group and Bacteroides acidifaciens and related species Parabacteroides goldsteinii when comparing WSTE vs. placebo. Bacteroides sp. have been suggested to reduce TMAO to TMA and the inventors therefore propose that a reduction in Bacteroidetes via WSTE is one mechanism explaining the observed effects on TMAO. The inventors also observed a reduction in Hungatella hathewayi and Rumonococcus faecis with WSTE. Hungatella hathewayi is known to be a TMA producer, while Rumonococcus was positively correlated with plasma TMAO in a study in rats. Finally, the inventors found an increase in Alistipes with WSTE, another member of the Bacteroidetes phylum.

### 3.4.3 the Impact of WSTE on Bile Acids

The inventors have noted that TMAO may promote atherosclerosis by several mechanisms, including inhibiting hepatic bile acid synthesis (Wang et al. (2011) Nature 472 p57-63). In a study in mice by Chen et al., (supra), resveratrol attenuated TMAO-induced atherosclerosis by inhibiting microbial TMA production and increasing hepatic bile acid synthesis. This was thought to be because resveratrol increased the relative abundance of Lactobacillus and Bifidobacterium, which resulted in an increased bile salt hydrolase activity, and bile acid deconjugation, and increased fecal excretion of bile. The resulting decrease in ileal bile acid content and repression of the enterohepatic farnesoid X receptor (FXR)-fibroblast growth factor 15 (FGF15) axis increased hepatic bile acid synthesis. The inventors found that plasma CDCA, GCA, GCDCA, and GDCA significantly increased in the WSTE group but not in the placebo group. This also suggests an increase in hepatic bile acid synthesis. However, the inventors did not observe changes in Lactobacillus or Bifidobacterium or reduced fecal bile acid contents.

### 3.4.4 the Impact of WSTE on LPS

WSTE also decreased plasma LPS, a gut microbiota-derived factor involved in the onset and progression of chronic inflammation-related diseases such as obesity, T2DM, or non-alcoholic fatty liver disease (NAFLD). Cani et al. ((2012) Gut Microbes 3 p279-288) showed that a high-fat diet contributes to the disruption of tight-junction proteins and that this effect was directly dependent on the gut microbiota because antibiotic treatment abolished diet-induced gut permeability. Moreover, they found that high-fat feeding augmented plasma LPS at a concentration sufficient to increase metabolic endotoxemia. Specific modulation of gut microbiota composition with prebiotics improved gut barrier integrity reduced metabolic endotoxemia and lowered inflammation and glucose intolerance. Interestingly, in patients with T2DM and advanced chronic kidney disease, TMAO showed a positive correlation with zonulin, a gut permeability marker, and LPS, as well as inflammatory (IL-6, TNFα, and CRP) and endothelial dysfunction (ET-1) biomarkers (Al-Obadie et al. (2017) J Clin Med6:E86) suggesting that zonulin upregulation leads to an uncontrolled influx of microbial endotoxins such as TMA and LPS trafficking from the intestine to the bloodstream driving low-grade inflammation and endothelial dysfunction.

Therefore, the observed reduction in plasma LPS following WSTE consumption suggests improvements in intestinal barrier function, and this data illustrates the usefulness of WSTE for treating or preventing chronic inflammation-related diseases such as IBS, IBD, obesity, T2DM, or non-alcoholic fatty liver disease (NAFLD).

### 3.4.5 Summary

The inventors found that 4 weeks of supplementation with WSTE (a watery tomato extract rich in secondary metabolites including polyphenols):
(A) decreased fasting blood and urine TMAO compared to baseline;
(B) decreased plasma LPS, a marker of intestinal permeability and low-grade inflammation;
(C) increased plasma levels of bile salts; and
(D) these changes were paralleled by changes in fecal microbiota with increases in Jaccard distance-based PC1, but not alpha diversity, and several changes in microbial composition such as decreases in Bacteroides, Ruminococccus, and Hungatella related OTUs, which are known for their involvement in TMA/TMAO metabolism, and increases in Alistipes and Clostridia related OTUs.

These data strongly support a use for WSTE for modulating the gut microbiota to confer host health benefits. The cardiovascular health benefits of WSTE are apparent from earlier publications made by the applicant (see WO 99/55350, WO 2010/049707 or WO 2018/083137). The data presented here may give a new insight into the mechanisms by which WSTE may contribute to its published effects on cardiovascular health. However, this data also illustrates that WSTE is unexpectedly useful as a prebiotic. As such it may be used for promoting gut health. This led the inventors to realise that: (a) WSTE may be advantageously formulated for delivery to the large intestine and, furthermore may be useful when co-administered with other prebiotics or with probiotics; and (b) the knowledge that WSTE modulates the gut microbiome, TMAO levels and LPS levels reveals new and unexpected uses for WSTE. These include treating chronic inflammatory-related diseases, type II diabetes, obesity and cognitive dysfunction.

1. A water-soluble tomato extract with activity for inhibiting platelet aggregation for use in modulating the gut microbiota to confer a health benefit in a subject.

2. The water-soluble tomato extract for use according to claim 1 wherein the extract acts as a prebiotic.

3. The water-soluble fruit extract for use according to claim 1 or 2 comprising a fraction of a tomato extract with activity for inhibiting platelet aggregation, the fraction containing a substantially heat stable colourless water soluble compound or compounds that have activity for preventing platelet aggregation and having a molecular weight of less than 1000 daltons.

4. The water-soluble tomato extract for use according to any one of claims 1-3 that:
(a) is substantially heat stable;
(b) is colourless or straw-coloured;
(c) is water soluble;
(d) consists of components having a molecular weight of less than 1000; and
(e) contains one or more compounds which inhibit platelet aggregation selected from:
(i) glycosylated phenolic acid or a phenolic ester, or derivatives thereof;
(ii) a glycosylated flavonoid; and
(iii) a nucleoside.

5. The water-soluble fruit extract for use according to claim 4 comprising each of: a glycosylated phenolic acid or a phenolic ester, or derivatives thereof; a glycosylated flavonoid; and a nucleoside.

6. A composition formulated for delivery of a water-soluble tomato extract with activity for inhibiting platelet aggregation to the large intestine of a subject comprising;
(a) a water-soluble extract of tomato fruit as defined by any one of claims 1-5; and
(b) at least one excipient that promotes delivery of the water-soluble tomato extract to the large intestine.

7. A composition comprising:
(a) a water-soluble extract of tomato fruit as defined by any one of claims 1-5; and
(b) a prebiotic.

8. A composition comprising:
(a) a water-soluble extract of tomato fruit as defined by any one of claims 1-5; and
(b) a probiotic.

9. The water-soluble extract for use according to any one of claims 1-5 or a composition according to one of claims 6-8 in the form of a pharmaceutical product, nutraceutical product, health supplement, drink, food substance or food supplement.

10. The water-soluble extract or composition of claim 9 formulated as a gel, powder, food bar, beverage, powder, tablet or contained within a capsule.

11. The use or composition as defined by any previous claim that is used to confer a health benefit to the digestive system of a subject.

12. The use or composition as defined by claim 11 that is used to confer a health benefit selected from improving digestion, improving resistance to infection in the gut, reducing inflammation in the gut, treating or minimising inflammatory Bowel Disease or treating or minimising irritable bowel syndrome.

13. The use or composition as defined by any one of claims 1-10 that is used to confer a health benefit in subjects with conditions whose pathology is caused or aggravated by compounds released by the gut microbiota into the bloodstream; and whereby the water-soluble tomato extract modulates plasma levels of such compounds by modulating the gut microbiota.

14. The use or composition as defined by claim 13 for conferring a health benefit in subjects suffering from anxiety, depression, Type II diabetes, non-alcoholic fatty liver disease, obesity or cognitive dysfunction.

15. A method of modulating the gut microbiota of a subject to confer a health benefit in the subject comprising administering a water-soluble tomato extract with activity for inhibiting platelet aggregation to a subject in need of such treatment.


SEQUENCE LISTING (2 sequences)

SEQ ID NO: 1 - DNA, 17 bp, synthetic construct
  source 1..17: mol_type = other DNA
cctacgggnggcwgcag

SEQ ID NO: 2 - DNA, 21 bp, synthetic construct
  source 1..21: mol_type = other DNA
gactachvgggtatctaatcc